(19) United States
(12) Patent Application Publication
(10) Pub. No.: US 2026/0251644 A1
(43) Pub. Date: Aug. 27, 2026

(54) NANOPLASMONIC SENSOR

(71) Applicant: Nanopath Inc., Cambridge, MA (US)

(72) Inventors: Amogha Tadimety, Cambridge, MA (US); Alison Burklund, Cambridge, MA (US); Timothy Palinski, Denver, CO (US); David Luna, Somerville, MA (US)

(21) Appl. No.: 18/875,037

(22) PCT Filed: Jun. 15, 2023
(86) PCT No.: PCT/US2023/025468

§ 371 Date: Dec. 13, 2024

Related U.S. Application Data
(60) Provisional application No. 63/352,970, filed on Jun. 16, 2022.
(60) Provisional application No. 63/352,972, filed on Jun. 16, 2022.

(51) Int. Cl. G01N 33/543; G01N 21/552; G01N 33/531

(52) U.S. Cl. CPC G01N 33/54373; G01N 21/554; G01N 33/531; G01N 2201/023

(57) ABSTRACT

This disclosure is related to the field of molecular detection. Specifically, the disclosure describes a rational nanoplasmonic sensor design for molecular detection. Also disclosed herein is a method of making a functionalized nanoplasmonic sensing chip. In some embodiments, the method comprises providing a substrate comprising an array of sensors, each sensor comprises an array of nanostructures; affixing a micro-well adaptor on top of the substrate, thereby providing an array of micro-wells over and aligned with the array of sensors; and forming one or more of functionalized sensors in the array of sensors by delivering a first batch of reaction solutions into one or more micro-wells atop one or more sensors using an automatic pipetting system; subsequently removing the first batch of reaction solution from the one or more micro-wells using the automatic pipetting system; and removing the microwell adaptor from the substrate.

### INCORPORATION BY REFERENCE TO ANY PRIORITY APPLICATIONS

This application claims priority to U.S. Provisional Patent Application No. 63/352,970, filed on Jun. 16, 2022 titled “Nanoplasmonic Sensor” and U.S. provisional Application No. 63/352,972, filed on Jun. 16, 2022, titled “Functionalized Nanoplasmonic Sensor.” Any and all applications, if any, for which a foreign or domestic priority claim are hereby incorporated by reference in their entireties.

### BACKGROUND

### Field of the Invention

This disclosure is related to the field of molecular detection. Specifically, the disclosure describes a method for functionalization of nanoplasmonic sensor and a functionalized nanoplasmonic sensor.

### Description of the Related Art

For biomedical research, clinical diagnostics, environmental testing, and other related fields, it is beneficial to have apparatuses and systems for detecting analytes, such as biomolecules and chemical substances with high accuracy, sensitivity, specificity, reproducibility, and ease of use. For example, having fast, rapid, and accurate tests for detecting certain analytes in a biological sample may aid in clinical diagnosis contexts, and assist physicians in determining optimal treatment regimens.

Metals have the unique ability to support electromagnetic surface waves, called surface plasmons, when optically illuminated. This property, and its strong sensitivity to changes in refractive index, allows for the use of metal nanostructures as highly sensitive transducers. In prior work described by our group, ensembles of randomly oriented nanostructures (i.e., colloidal nanorods dispersed on chip) were employed for sequence-specific nucleic acid sensing. While these particle sensors have the advantage of rapid, instrument-free fabrication, they suffer from low sensitivity and quality factor due to the random particle dispersity.

### SUMMARY

Disclosed herein is a plasmon-resonance sensing device. In some embodiments, the plasmon-resonance sensing device comprises: (1) an array of sensors, each sensor comprises an array of nanostructures that are regularly spaced apart with a spacing of from about 100 nm and about 2000 nm between the nanostructures, (2) wherein each of the nanostructures has a square shape with a side dimension of from about 50 nm to about 400 nm, and (3) the nanostructures are conjugated with a biological probe configured to bind to an analyte. In some embodiments, the nanostructures have a thickness of from about 20 nm to about 75 nm. In some embodiments, the nanostructures comprise gold. In some embodiments, the biological probe is selected from the group consisting of a peptide-nucleic acid, an aptamer, an antibody, an antibody fragment, a complimentary DNA, and an enzyme. In some embodiments, at least a first sensor in the array of sensors comprises nanostructures conjugated with a first biological probe and at least a second sensor in the array of sensors comprises nanostructures conjugated with a second biological probe.

Also disclosed herein is a method for detecting an analyte in a sample. In some embodiments, the method comprises (1) exposing at least one sensor in the plasmon-resonance sensing device of claim 1 to the sample, wherein the analyte in the sample binds to the at least one sensor, (2) illuminating a light at a series of wavelengths onto the at least one sensor, and (3) collecting absorbance, transmittance, or extinction data of the sensor. In some embodiments, the method further comprising heating the at least one sensor after exposing the at least one sensor to the sample. In some embodiments, the method further comprises comparing the collected absorbance, transmittance, or extinction data with a baseline data of the sensor prior to the sample exposure. In some embodiments, an array of sensors in the plasmon-resonance sensing device of any of the present disclosures is exposed to the sample. In some embodiments, at least a first sensor in the array of sensors comprises nanostructures conjugated with a first biological probe and at least a second sensor in the array of sensors comprises nanostructures conjugated with a second biological probe. In some embodiments, the first biological probe and the second biological probe are independently selected from the group consisting of a protein-nucleic acid, an aptamer, an antibody, an antibody fragment, a complimentary DNA, and an enzyme. In some embodiments, the first biological probe and the second biological probe are different.

In some embodiments, the method further comprising flowing a plurality of functionalizes particles over the at least one sensor after exposing the at least one sensor to the sample, wherein the plurality of functionalized particles is configured to bind to the analyte that is bound to the at least one sensor

Also disclosed herein is a method of making an array of nanostructures. In some embodiments, the method comprises: (1) coating a conductive photoresist layer onto a non-conductive substrate, (2) patterning the conductive photoresist layer via lithography thereby forming a patterned substrate, (3) depositing an adhesion layer onto the patterned substrate, and (4) depositing a metallic layer onto the adhesion layer. In some embodiments, the metallic layer comprises gold. In some embodiments, the metallic layer has a thickness of about 20 nm to about 75 nm. In some embodiments, the adhesion layer comprises chromium. In some embodiments, the adhesion layer has a thickness of about 5 nm. In some embodiments, the nanostructures are regularly spaced apart with a spacing of from about 100 nm and about 2000 nm in between, and each of the nanostructures has a square shape with a side dimension of from about 50 nm to about 400 nm.

Disclosed herein is a method of making a functionalized nanoplasmonic sensing chip. In some embodiments, the method comprises (1) providing a substrate comprising an array of sensors, each sensor comprises an array of nanostructures, (2) affixing a micro-well adaptor on top of the substrate, thereby providing an array of micro-wells over and aligned with the array of sensors, (3) forming one or more functionalized sensors in the array of sensors, and (4) removing the micro-well adaptor from the substrate. forming one or more functionalized sensors in the array of sensors includes delivering a first batch of reaction solutions into one or more micro-wells atop one or more sensors, subsequently removing the first batch of reaction solutions from the one or more micro-wells, wherein the delivering and removing of the first batch of reaction solutions are performed by an automatic pipetting system. In some embodiments, forming one or more functionalized sensors further comprises delivering a second batch of reaction solutions into the one or more micro-wells, and subsequently removing the second batch of reaction solutions from the one or more micro-wells, wherein the delivering and removing of the second batch of reaction solutions are performed by the automatic pipetting system.

In some embodiments, the one or more functionalized sensors comprises one or more biological probes. In some embodiments, each of the one or more biological probe are independently selected from the group consisting of a peptide-nucleic acid, an oligonucleotide, an aptamer, an antibody, an antibody fragment, a complimentary DNA, and an enzyme. In some embodiments, the one or more biological probes are the same. In some embodiments, all of the one or more biological probes are different. In some embodiments, the first batch of reaction solutions are delivered to the one or more micro-wells simultaneously.

Also disclosed herein is a functionalized nanoplasmonic sensing chip. In some embodiments, the functionalized nanoplasmonic sensing chip comprises an array of functionalized sensors. In some embodiments, each of the functionalized sensors in the array comprises an array of nanostructures conjugated to at least one biological probe configured to bind to at least one analyte. In some embodiments, the array of nanostructures is conjugated to two or more biological probes configured to bind to two or more analytes. In some embodiments, the at least one biological probe is independently selected from the group consisting of a peptide-nucleic acid, an oligonucleotide, an aptamer, an antibody, an antibody fragment, a complimentary DNA, and an enzyme. In some embodiments, at least one of the functionalized sensors in the array comprises at least one different biological probe from the others. In some embodiments, each of the functionalized sensors in the array comprises at least one different biological probe. In some embodiments, the nanostructures comprise gold. In some embodiments, the nanostructures in the array are regularly spaced apart with a spacing of from about 100 nm and about 2000 nm, and each nanostructure has a square shape with a side dimension of from about 50 nm to about 400 nm. In some embodiments, the nanostructures have a thickness of from about 20 nm to about 75 nm.

Also disclosed herein is a method for detecting two or more analytes simultaneously. In some embodiments, the method comprises exposing the array of functionalized sensors on the functionalized nanoplasmonic sensing chip of any of the present embodiments to the sample, illuminating a light at a series of wavelengths onto each of the functionalized sensors, and collecting absorbance, transmittance, or extinction data of each functionalized sensor. In some embodiments, the method further comprises comparing the collected absorbance, transmittance, or extinction data of each functionalized sensor with a baseline data of each of the functionalized sensor prior to the sample exposure. In some embodiments, the method further comprising heating the array of functionalized sensors after exposing the array of functionalized sensors to the sample. In some embodiments, up to 50 analytes in the sample are detected.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A depict one embodiment of a plasmonic-resonance sensing device.

FIG. 1B depicts one embodiment of an array of nanostructures in a sensor of the plasmonic-resonance sensing device.

FIGS. 2A-2B depict non-limiting example schematics of selected geometries and fabrication maps. FIG. 1A illustrates a schematic of a grid with labeled dimensions for length, width, thickness, and periodicity of nanostructures. FIG. 1B illustrates a schematic of a map of arrangement of dimensions for dose matrix test.

FIG. 3 shows extinction curves of a non-limiting example of regular gold nanorod array at three bulk refractive indices.

FIGS. 4A-4B depict examples of PNA-DNA Binding Simulations. The simulations are of conformal layers representing PNA and DNA binding to gold nanostructure. The two geometries demonstrated here are (FIG. 3A) repeating nanorod array (130 nm×40 nm) and (FIG. 3B) repeating nanosquare array (95 nm×95 nm).

FIG. 5A shows the extinction curve for bulk refractive index sensitivity simulations in one embodiment of the nanostructure arrays at three refractive indexes. FIG. 5B depicts the refractive index sensitivities of uncoupled nanorods and the nanostructure array.

FIG. 6A shows the extinction curve for bulk refractive index sensitivity simulations in one embodiment of the nanostructure arrays at three refractive indexes. FIG. 6B depicts the refractive index sensitivities of uncoupled nanorods and the nanostructure array.

FIG. 7A shows the extinction curve for bulk refractive index sensitivity simulations in one embodiment of the nanostructure arrays at three refractive indexes. FIG. 7B depicts the refractive index sensitivities of uncoupled nanorods and the nanostructure array.

FIG. 8 depicts the experimental transmission spectra for 5 different nanoarray geometries.

FIG. 9 depicts the simulated transmission spectra for each 5 different nanoarray geometries.

FIG. 10 depicts a CAD drawing of post array polymer well mold and fabricated well, with coordinates aligned over the sensor array.

FIGS. 11A-11B depict two alternative views of a 3D printed mold for a fabricated polymer well.

FIGS. 11C-11D depict two alternative views of the final fabricated well array, made from the mold of FIGS. 11A and 11B.

FIGS. 11E-11I depict additional embodiments of micro-well fixtures.

FIGS. 12A-C depict one embodiment of the automatic pipette system. FIG. 12A depicts the overall system, with pipette holder on the left, tip box, 96-well plate holder, and custom chip adapter. FIG. 12B depicts the tip box aligned under pipette holder. FIG. 12C depicts the 96 well plate and adapter during functionalization.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

A plasmon-resonance sensing device employing ordered array nanostructure ensembles is described herein. The ordered array of nanostructures allows for coupling to diffractive photonic modes, which can be used to improve sensor sensitivity. The nanostructure dimension and geometry arc tailored to provide high quality signal and large optical shifts upon modeled analyte binding.

The present disclosure generally relates to a plasmon-resonance sensing device employing ordered array nanostructure ensembles and the method for detecting an analyte using the plasmon-resonance sensing device. Also disclosed herein are a method of making the nanostructure array and the use of full-wave electromagnetic simulations coupled to experiments for design of nanoplasmonic arrays for biosensing. The ordered array of nanostructures allows for coupling to diffractive photonic modes, which results in improved sensor sensitivity. The nanostructure dimension and geometry are tailored to provide high quality signal and large optical shifts upon modeled analyte binding. The plasmon-resonance sending device disclosed herein involves rational design of sensor arrays for nanoplasmonic transduction of analyte binding. In some embodiments, the nanoparticle array geometries may be utilized for the detection of DNA sequences. The nanostructure array geometry design may afford high sensitivity and quality factor biosensing.

### Plasmon-Resonance Sensing Devices

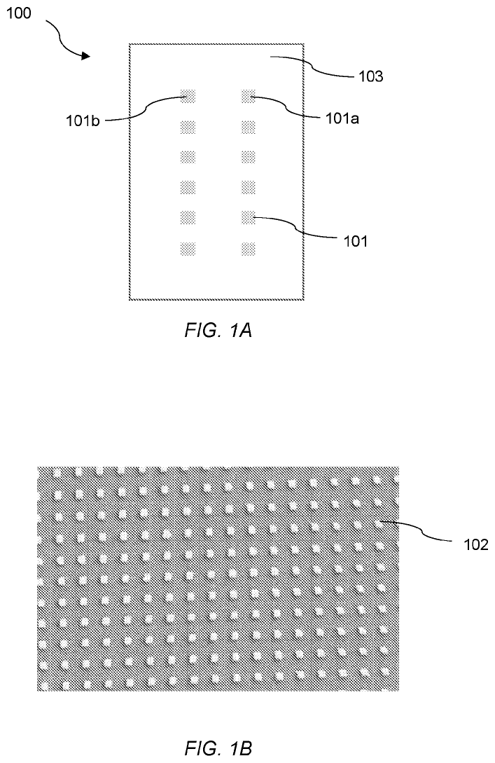

Disclosed herein is a plasmon-resonance sensing device. As shown in FIGS. 1A and 1B, the plasmon-resonance sensing device 100 comprises an array of sensors 101. Each sensor 101 comprises an array of nanostructures 102 that are regularly spaced apart. In some embodiments, the nanostructures 102 are regularly spaced apart with a spacing of about 100 nm, about 200 nm, about 300 nm, about 500 nm, about 750 nm, about 1000 nm, about 1200 nm, about 1500 nm, about 1800 nm, about 2000 nm, or any distance that is between about 100 nm and about 2000 nm, between the nanostructures. In some embodiments, the array of nanostructures are regularly spaced apart with a spacing of from about 100 nm to about 2000 nm, from about 100 nm to about 1800 nm, from about 100 nm to about 1600 nm, from about 100 nm to about 1400 nm, from about 100 nm to about 1200 nm, from about 100 nm to about 1000 nm, from about 200 nm to about 900 nm, from about 300 nm to about 800 nm, from about 100 nm to about 400 nm, from about 200 nm to about 500 nm, from about 300 nm to about 600 nm, from about 400 nm to about 700 nm, from about 500 nm to about 800 nm, from about 600 nm to about 900 nm, from about 700 nm to about 1000 nm, from about 500 nm to about 2000 nm, or from about 500 nm to about 1500 nm between the nanostructures.

The nanostructures in the array may have various shapes. For example, the nanostructures may have a rectangular shape, a circular shape, a triangular shape, a star shape, a pentagon shape, a parallelogram shape, a diamond shape, or a square shape. Preferably, each of the nanostructures in the array has a square shape. In some embodiments, each nanostructure has a side dimension of about 50 nm, about 75 nm, about 100 nm, about 150 nm, about 200 nm, about 250 nm, about 300 nm, about 350 nm, or about 400 nm, or any integer that is between about 50 to about 400 nm. In some embodiments, the square shape has a side dimension of from about 50 nm to about 400 nm, from about 100 nm to about 350 nm, from 150 nm to about 300 nm, from about 50 nm to about 150 nm, from about 100 nm to about 200 nm, from 150 nm to about 250 nm, from about 200 nm to about 300 nm, from about 250 nm to about 350 nm, or from about 300 nm to about 400 nm, or any range that is between about 50 nm and about 400 nm.

In some embodiments, the nanostructures in the array may have a thickness of about 20 nm, about 25 nm, about 30 nm, about 35 nm, about 40 nm, about 45 nm, about 50 nm, about 60 nm, about 65 nm, about 70 nm, about 75 nm, or any integer between about 20 and about 75 nm. In some embodiments, the nanostructures in the array may have a thickness of from about 20 nm to about 75 nm, from about 25 nm to about 70 nm, from about 30 nm to about 65 nm, from about 35 nm to about 60 nm, from about 30 nm to about 55 nm, or any range that is between about 20 and about 75 nm.

The nanostructures comprise a metal. For example, the nanostructures may comprise gold, platinum, aluminum, silver, or copper. Preferably, the nanostructure comprises gold. In some embodiments, the nanostructures comprise a single metal. In some embodiments, the nanostructures comprise a mixture of metals.

In some embodiments, the nanostructures in the array are conjugated with a biological probe. The biological probe is configured to bind to an analyte. The binding of the analyte to the biological probe alters the surface properties of the nanostructure, thereby causing a change in localized surface plasmon resonance. In some embodiments, the biological probe comprises one or more of a protein, peptide strand, amino acid, RNA strand, DNA strand, or and/or nucleotide. In some embodiments, the biological probe comprises one or more of a modified protein, modified peptide, modified amino acid, modified RNA strand, modified DNA strand, and/or modified nucleotide. In some embodiments, the biological probe comprises at least one of: a peptide-nucleic acid, an aptamer, an antibody, an antibody fragment, a complimentary DNA, and/or an enzyme. In some embodiments, the biological probe is selected from the group consisting of a peptide-nucleic acid, an aptamer, an antibody, an antibody fragment, a complimentary DNA, and an enzyme.

In some embodiments, at least a first sensor 101a in the array of sensors comprises nanostructures 102 conjugated with a first biological probe. In some embodiments, at least a second sensor 101b in the array of sensors comprises nanostructures conjugated with a second biological probe. In some embodiments, at least a third sensor in the array of sensors comprises nanostructures conjugated with a third biological probe. In some embodiments, at least a fourth sensor in the array of sensors comprises nanostructures conjugated with a fourth biological probe. In some embodiments, at least a fifth sensor in the array of sensors comprises nanostructures conjugated with a fifth biological probe. In some embodiments, a “n” number of sensors in an array of sensors comprises nanostructures conjugated to an “n” number of biological probes, wherein “n” is any number from 1 to 2000 In some embodiments, 6 or 12 sensors may be presented in the array of sensors on a substrate 103. In some embodiments, the sensors may have an area of from about 1 μm² to about 1 mm². In some embodiments, the sensors may have an area of from about 10 μm² to about 1 mm², about 50 μm² to about 1 mm², about 100 μm² to about 1 mm², about 200 μm² to about 1 mm², about 400 μm² to about 1 mm², or about 500 μm² to about 1 mm².

The substrate 103 may be a dielectric or non-conductive substrate. In some embodiments, the substrate 103 is transparent to allow the sensors to be exposed to the incident light through the substrate 103. For example, the substrate 103 may be a glass substrate, a plastic substrate, or a polymeric substrate. The substrate and the sensor array on the substrate may be integrated with a microfluidic module to provide a means for introducing or exposing the sample to the sensors.

### Analyte Detection

Disclosed herein is a method for detecting an analyte in a sample. In some embodiments, the method comprises exposing at least one sensor 101 in the plasmon-resonance sensing device 100 of any of the embodiments disclosed herein to a sample. The sample may or may not comprise the target analyte. The plasmon-resonance sensing device 100 can be utilized to detect the presence of an analyte (i.e., a target analyte). In some embodiments, the method comprises exposing at least two sensors in the plasmon-resonance sensing device 100 of any of the embodiments disclosed herein to a sample. In some embodiments, the method comprises exposing at least three sensors, at least four sensors, at least 5 sensors, or at least 6 sensors in the plasmon-resonance sensing device 100 of any of the embodiments disclosed herein to a sample. In some embodiments, the method comprises exposing an “n” number of sensors in the plasmon-resonance sensing device of any of the embodiments disclosed herein to a sample, wherein “n” is any number from 1 to 2000. In some embodiments, the array of sensors is exposed to the sample. The sample may comprise a bodily fluid, such as blood, plasma, mucus, serum, urine, or saliva, etc. Mucus can be collected via cervical swabs, vaginal swabs, or nasal swabs. When the at least one sensor 101 is exposed to the sample, the biological probe in each sensor would selectively bind to the analyte that the biological probe is configured to bine.

Optionally, the at least one sensor may be subject to a heating step after the exposure to the sample. In some embodiments, the at least one sensor is heated up to about 85° C. or any temperature between 25° C. and 85° C. In some embodiments, the at least one sensor may be exposed to heat before, during, or after subsequent steps. In some embodiments, the at least one sensor may be exposed to heat before, during, or after the measurement.

The method for detecting or sensing an analyte further comprises illuminating a light onto the at least one sensor. In some embodiments, the method comprises illuminating a light at a series of wavelengths onto the at least one sensor. In some embodiments, the light may be emitted from a light source in an apparatus for analyte detection. The light source may be configured to emit a series of wavelengths for illuminating the sensor. In some embodiments, the plasmonic sensing chip containing the sensors may be inserted into the apparatus for analyte detection. The apparatus is configured to emit a light at a series of wavelengths onto the sensors, and to collect an optical spectrum of the light transmitted through, absorbed by, or reflected from the sensors. For example, the apparatus can perform absorbance/transmittance measurements. In some embodiments the measurements are made at wavelengths ranging from 500-1000 nm.

The method further comprises collecting data from the sensor. In some embodiments, the method comprises collecting absorbance data from the sensor. In some embodiments, the method comprises collecting transmittance data from the sensor. In some embodiments, the method comprises collecting extinction data from the sensor. In some embodiments, the method comprises collecting absorbance, transmittance, and/or extinction data of the sensor. In some embodiments, the method further comprises comparing collected data with a baseline data of the sensor prior to the sample exposure. In some embodiments, the method further comprises comparing at least one of the collected absorbance, transmittance, and/or extinction data with a baseline data of the sensor prior to the sample exposure. For example, the absorbance/transmittance measurements of functionalized sensors are made prior to exposure to the sample. The peak absorbance wavelength of the functionalized sensor (prior to bonding with a target analyte) is identified. The absorbance/transmittance of the sensors are made again after exposing to the sample, and a shift in peak absorbance can be observed if a target analyte is present in the sample and binds with the probe on the functionalized sensors. The shift represents the detection signal.

In some embodiments, an array of sensors in the plasmon-resonance sensing device 100 of any of the present embodiments is exposed to the sample. In some embodiments, at least a first sensor 101a in the array of sensors 101 comprises nanostructures conjugated with a first biological probe. In some embodiments, at least a second sensor 101b in the array of sensors 101 comprises nanostructures conjugated with a second biological probe. In some embodiments, at least a third sensor in the array of sensors comprises nanostructures conjugated with a third biological probe. In some embodiments, at least a fourth sensor in the array of sensors comprises nanostructures conjugated with a fourth biological probe. In some embodiments, at least a fifth sensor in the array of sensors comprises nanostructures conjugated with a fifth biological probe. In some embodiments, a “n” number of sensors in an array of sensors comprises nanostructures conjugated to an “n” number of biological probes, wherein “n” is any number from 1 to 2000. The biological probes conjugated to different sensors may be the same or different. In some embodiments, each sensor in the array can be conjugated to different biological probes for a multiplex sensing capability. In this configuration, multiple analytes can be detected simultaneously.

In some embodiments, at least a first sensor 101a in the array of sensors comprises nanostructures conjugated with a first biological probe and at least a second sensor 101b in the array of sensors comprises nanostructures conjugated with a second biological probe. In some embodiments, a first set of sensors in the sensor array is functionalized with a first biological probe, and a second set of sensors in the sensor array is functionalized with a second biological probe. In some embodiments, the first biological probe and the second biological probe are different. In some embodiments, the first biological probe and the second biological probe are the same. In some embodiments, the first biological probe and the second biological probe independently comprise one or more of a protein, peptide strand, amino acid, RNA strand, DNA strand, or and/or nucleotide. In some embodiments, the first biological probe and the second biological probe independently comprise one or more of a modified protein, modified peptide, modified amino acid, modified RNA strand, modified DNA strand, and/or modified nucleotide. In some embodiments, the first biological probe and the second biological probe independently comprise at least one of: a peptide-nucleic acid, an aptamer, an antibody, an antibody fragment, a complimentary DNA, and/or an enzyme. In some embodiments, first biological probe and the second biological probe are independently selected from the group consisting of a peptide-nucleic acid, an aptamer, an antibody, an antibody fragment, a complimentary DNA, and an enzyme.

The detection of analyte(s) is based on an optical phenomenon that occurs between a metal nanostructure and a dielectric-localized surface plasmon resonance (LSPR). LSPR is observed when the wavelength of incident light is larger than the size of the conductive nanostructures. The nanostructures result in highly confined electric fields of LSPR modes, which serve as a sensitive transducer to changes in the local dielectric environment (binding event). The nanostructures can be conjugated to/covalently functionalized with probes that can bind with target analytes. Upon binding with the target analyte(s), red shifts in the spectral peak can be observed. In some embodiments the amount of red shift may be observed as a function of target analyte concentration. In some embodiments, the sensors detect transmittance, reflectance, and/or absorbance at certain wavelength range.

In some embodiments, the sensors that have been exposed to the sample, thus having analyte(s) bound to selective biological probe on the sensors, can be further exposed to functionalized particles configured to bind to the sensors that have analyte(s) present and bound to the biological probe. The functionalize particles may be nanoparticles or microparticles. In some embodiments, the particles may be metal, polymer, glass, or any material with a high refractive index, for example, a refractive index of about 1.5 and higher. When the functionalized particles are bound to the sensors, it has the potential to improve both sensitivity and specificity of the sensors. Without being bound to the theory, the sensitivity improvements may be due to the fact that the functionalized particle increases the change in refractive index at the sensor surface in the presence of the analyte. The additional binding of the functionalized particles to the sensors may improve the sensor signal through a greater peak-shift in the optical measurement. Specificity improvements may be due to the fact that two selective binding events are required (i.e., first analyte must bind to the sensor, then the functionalized particle must bind to the sensor-bound analyte). In some embodiments, the functionalized particles are functionalized to bind to the analytes that have bound to the biological probes.

In some embodiments, a spectrum of the sensor comprising an array of functionalized nanostructures may be obtained prior to exposure to a sample. This may provide baseline data for the determination and analysis of an analyte binding event.

### Nanostructures Fabrication

Also disclosed herein is a method of making an array of nanostructures. The method comprises coating a photoresist layer onto a substrate, patterning the photoresist, and depositing a metallic layer over the patterned photoresist layer. In some embodiments, the substrate may be non-conductive, and a modified method may provide an improved result. The method comprises coating a conductive photoresist layer onto a non-conductive substrate, patterning the conductive photoresist layer via photolithography, depositing an adhesion layer over the patterned conductive photoresist layer, and depositing a metallic layer onto the adhesion layer. In some embodiments, patterning the conductive photoresist layer comprises exposing the photoresist layer to the electron beam to create a desired pattern. In some embodiments, the pattern should match the dimensions of and the spacing between the nanostructures. In some embodiments, the method may involve lithographic techniques, such as electron-beam lithography, UV photolithography, or nanoimprint lithography. In some embodiments, roll-to-roll manufacturing may be employed for making the sensor array.

For example, photolithography may be utilized to remove the portions of the photoresist layer where the nanostructures should be disposed/formed on the substrate, leaving the portion of the substrate where there should not be any nanostructure masked by the patterned photoresist layer. The patterned photoresist layer therefore has removed portions resembling the size, shape, and location of where the metallic nanostructures should be disposed. The portion of substrate is exposed at where the nanostructures will be formed. When the metallic layer is subsequently disposed over the patterned photoresist layer, some metallic layer would be disposed on the exposed portions of the substrate, and some the metallic layer would be disposed on the remaining photoresist that is masking the substrate.

The method further comprises lifting off the patterned photoresist layer. Lifting off the patterned photoresist layer also takes off the portions of the adhesive layer and the metallic layer disposed on the remaining patterned photoresist layer, leaving behind the portions of the adhesive layer that are in contact with the substrate and the portions of the metallic layer on that portions of the adhesive layer. In some embodiments, the adhesion layer comprises chromium. In some embodiments, the adhesion layer has a thickness of about 2 nm, about 3 nm, about 4 nm, about 5 nm, about 6 nm, about 8 nm, about 9 nm, or any thickness that is between about 2 and about 9 nm. In some embodiments, the adhesion layer has a thickness of about 5 nm. In some embodiments, the metallic layer comprises a single metal. In some embodiments, the metallic layer comprises a mixture of metals. In some embodiments, the metallic layer comprises gold, silver, aluminum, platinum or copper. In some embodiments, the metallic layer comprises gold. The thickness of the metallic layer would be the same as the thickness of the nanostructures on the substrate as disclosed herein.

The method disclosed herein provides an array of sensors comprising an array of nanostructures that are regularly spaced apart. The shape, dimensions, and the spacing of the nanostructures made by such method are the same as disclosed herein.

### Functionalization of Nanoplasmonic Sensing Chip

Disclosed herein is a method of making a functionalized nanoplasmonic sensing chip. The method comprises providing a substrate comprising an array of sensors, affixing a micro-well adaptor on top of the substrate so an array of micro-wells is over the array of sensors and aligned with each sensor, and forming one or more functionalized sensors in the array of sensors. Forming the one or more functionalized sensors includes delivering a first batch of reaction solutions into one or more micro-wells atop one or more sensors using an automatic pipetting system, and then subsequently removing the first batch of reaction solution from the one or more micro-wells using the automatic pipetting system. The automatic pipetting system includes an array of pipets that can be loaded with one or more reaction solutions. In some embodiments, the array of pipets may be loaded with two or more different reaction solutions, thus allowing delivery of two or more different reaction solutions to the array of micro-wells/sensors. The array of pipets may also be used to remove the reaction solutions from some or all of the micro-wells/sensors after the reactions. The array of pipets can deliver or remove reaction solutions from a specific micro-well/sensor or a specific group of micro-wells/sensors. In some embodiments, each reaction solution may include one or more reagents for modifying the array of nanostructures in the sensor. In some embodiments, each reaction solution may include one or more biological probes.

In some embodiments, multi-step reactions may be utilized for functionalizing the sensors. Thus, forming one or more functionalized sensors may further involve delivering a second batch of reaction solutions into the one or more micro-wells, and subsequently removing the second batch of reaction solutions from the one or more micro-wells, wherein the delivering and removing the second batch of reaction solutions are performed by an automatic pipetting system.

In some embodiments, the first batch of reaction solutions comprises two or more different reaction solutions. In some embodiments, the second batch of reaction solutions may also comprise two or more different reaction solutions. In some embodiments, the reaction solutions may include different biological probes. Thus the array of functionalized sensors may comprise two or more different biological probes. For example, some of the functionalized sensors in the array may comprise a specific biological probe, while other functionalized sensors comprise a different biological probe. In some embodiments, each of the functionalized sensor may comprise different biological probes. In some embodiments, a reaction solution may include one or more biological sensors. Thus each functionalized sensor may comprise one or more biological probes. One or more biological probes can conjugate to the array of nanostructures in each sensor. In some embodiments, the sensor may comprise one, two, three, four, or more biological probes configured to bind to one or more analytes.

Then the method further includes removing the micro-well adaptor from the substrate. In some embodiments, the one or more sensors are functionalized with a biological probe while the first batch of reaction solutions in the one or more micro-wells is in contact with the sensors. In some embodiments, the one or more sensors is functionalized with a biological probe after two or more reaction steps. In some embodiments, the sensor (e.g., the one or more sensors) each comprises an array of nanostructures disclosed herein.

In some embodiments, the automatic pipetting system can be configured to deliver different reaction solutions to multiple micro-wells for functionalizing multiple sensors in the array. In some embodiments, multiple reaction solutions are delivered to different sensors in the array, thereby functionalizing multiple sensors substantially at the same time. In some embodiments, the automatic pipetting system can be configured to removing different reaction solutions from multiple micro-wells. In some embodiments, multiple reaction solutions are removed from different sensors in the array substantially at the same time. In other embodiments, some reaction solutions may be removed at a different time to allow longer or shorter reaction time.

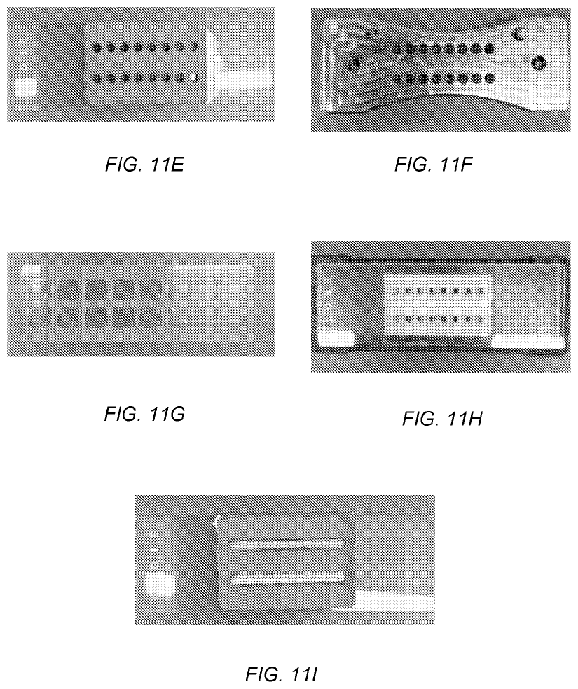

FIGS. 11A-11B depict two alternative views of a 3D printed mold for a fabricated polymer well shown in FIGS. 11C-11D. Other embodiments of the micro-wells are shown in FIGS. 11E-11I.

In some embodiments, additional pre-treatment step(s) can be performed prior to delivering any reaction solution. The pre-treatment step may include washing the nanostructure surface, wetting the nanostructure surface, or activation the nanostructure for subsequent reaction/functionalization. In some embodiments, the method may further comprise delivering an activation solution into at least a portion of the micro-wells atop the sensors in the array using an automatic pipetting system; and subsequently removing the activation solution prior to delivering a reaction solution.

The method disclosed herein provides at least one functionalized sensor comprises an at least one biological probe. In some alternatives, the first functionalized sensor comprises a first array of nanostructures conjugated to a first biological probe. In some alternatives, the second functionalized sensor comprises a second array of nanostructures conjugated to a second biological probe. In some alternatives, additional sensors comprising a nanostructures array may be conjugated to additional biological probe(s), up to the number of sensors in the sensor array. For example, a “n” number of sensors in an array of sensors comprises nanostructures conjugated to an “n” number of biological probes, wherein “n” is any number from 1 to 2000. In some embodiments, n may be any number from 1 to 1000, from 1 to 500, from 1 to 100, or from 1 to 25.

Each of the biological probes is independently selected from the group consisting of a peptide-nucleic acid (PNA), an oligonucleotide, an aptamer, an antibody, an antibody fragment, a complimentary DNA, and an enzyme. In some alternatives, the first biological probe and the second biological probe are independently selected from the group consisting of a peptide-nucleic acid, an aptamer, an antibody, an antibody fragment, a complimentary DNA, and an enzyme. In some alternatives, the first biological probe and the second biological probe are different. In some alternatives, the first biological probe and the second biological probe are the same. In some embodiments, each sensor may be functionalized with a different biological probe. In some embodiments, some of the sensors in the array may be functionalized with different biological probes. In some embodiments, all the sensors in the array may be functionalized with the same biological probe.

In some embodiments, reaction solutions are delivered to all the micro-wells simultaneously. In some alternatives, reaction solutions are subsequently removed from the micro-wells simultaneously. In some alternatives, reaction solutions are removed from the micro-wells at a different time to accommodate for different reaction time for functionalizing the sensors with a variety of the biological probes. In some embodiments, reaction solutions can also be delivered to different micro-wells at a different time. In some alternatives, the first reaction solution and the second reaction solution are delivered to the first micro-well and the second micro-well simultaneously, and subsequently the first reaction solution and the second reaction solution are removed from the first micro-well and the second micro-well. In some embodiments, delivering and removing a reaction solution may be performed by an automatic pipetting system. In some embodiments, the automatic pipetting system may be configured to remove different reaction solutions at a different time. In some embodiments, the automatic pipetting system may be configured to deliver different reaction solution at a different time.

In some embodiments, the nanostructures comprise a metal. In some alternatives, the nanostructures comprise a single metal. In some alternatives, the nanostructures comprise a mixture of metals. In some alternatives, the nanostructures comprise gold, platinum, aluminum, silver, or copper. In some alternatives, the nanostructures comprise gold.

### Functionalized Plasmonic Sensing Chip

Functionalized plasmonic sensing chips comprising an array of functionalized sensors are disclosed. In some embodiments, each of the functionalized sensors in the array comprises an array of nanostructures conjugated to at least one biological probe. In some embodiments, the array of nanostructures is conjugated to two or more biological probes configured to bind to two or more analytes. The biological probe is configured to bind to at least one analyte. In some alternatives, the at least one biological probe independently comprises at least one of: a peptide-nucleic acid, an oligonucleotide, an aptamer, an antibody, an antibody fragment, a complimentary DNA, and/or an enzyme. In some embodiments, the biological probe is independently selected from the group consisting of a peptide-nucleic acid, an oligonucleotide, an aptamer, an antibody, an antibody fragment, a complimentary DNA, and an enzyme. In some embodiments, all functionalized sensors in the array comprise the same biological probes. In some alternatives, at least one of the functionalized sensors in the array comprises at least one different biological probe from the others. For example, some of the functionalized sensors in the array may comprise a specific biological probe, while other functionalized sensors comprise a different biological probe. In some embodiments, each of the functionalized sensors in the array comprise at least one different biological probe. One or more biological probes can conjugate to the array of nanostructures in each sensor. In some embodiments, the functionalized sensor may comprise one, two, three, four, or more biological probes configured to bind to one or more analytes.

In some embodiments, the functionalized plasmonic sensor chip may include 1 to 100 (and any numbers in between) different biological probes. For example, the functionalized plasmonic sensor chip may include 1, 2, 3, 4, 5, 6, 7, 8, 9, 10, 11, 12, 13, 14, 15, 20, 24, 30, 36, 40, 48, 50, 54, 60, 70, 80, 90, or 100 different biological probes. In some embodiments, each functionalized sensor in the functionalized plasmonic sensor chip may contain different biological probe(s). In some embodiments, the array of the nanostructures in each sensor may conjugate to one or more biological probes, and the one or more biological probes may be different.

In some embodiments, the nanostructures comprise a metal. In some alternatives, the nanostructures comprise a single metal. In some alternatives, the nanostructures comprise a mixture of metals. In some alternatives, the nanostructures may comprise gold, platinum, aluminum, silver, or copper. In some alternatives, the nanostructures comprise gold. In some alternatives, the nanostructures in the array are regularly spaced apart and may have the geometry described herein.

### Multiplex Analyte Detections

A method for detecting two or more analytes simultaneously is also described. In some alternatives, the method may detect 3, 4, 5, 6, 7, 8, 9, 10, 11, 12, 13, 14, 15, 20, 24, 30, 36, 40, 48, 50, 54, 60, 70, 80, 90, and/or 100 analytes. In some embodiments, up to 50 analytes are detected. In some embodiments, up to 24, up to 50, up to 80, or up to 100 analytes may be detected. The method comprises exposing the array of functionalized sensors on the plasmonic sensing chip of any of the alternatives disclosed herein to a sample. The functionalized sensors are configured to detect the presence of certain target analytes. In some embodiments, the functionalized sensor may be configured to identify or detect various markers, subtypes, strains, genotypes and/or variants of a biological species. When the functionalized sensors are exposed to the sample, one or more target analytes, if present, bind to the corresponding biological probes. The binding event causes a change in the local dielectric environment of the sensors. The sample may comprise a bodily fluid, such as blood, urine, or saliva, etc. In some embodiments, the sample may be evacuated or removed from the functionalized sensors following the exposure step.

Optionally, the array of functionalized sensors may be subject to a heating step after the exposure to the sample. In some embodiments, the array of functionalized sensors is heated up to about 85° C. or any temperature between 25° C. and 85° C. In some embodiments, the array of functionalized sensors may be exposed to heat before, during, or after subsequent steps. In some embodiments, the array of functionalized sensors may be exposed to heat before, during, or after the measurement.

The method further comprises illuminating a light at a series of wavelengths onto the functionalized sensors; and collecting absorbance, transmittance, and/or extinction data from the functionalized sensors. The light may be emitted from a light source in an apparatus for analyte detection. The light source may be configured to emit a series of wavelengths for illuminating the sensors. In some embodiments, the plasmonic sensing chip containing the functionalized sensors may be inserted into the apparatus for analyte detection. The apparatus is configured to emit a light at a series of wavelengths onto the functionalized sensors, and to collect an optical spectrum of the light transmitted through, absorbed by, or reflected from the sensors.

In some embodiments, the method further comprises comparing collected data with a baseline data of the sensors prior to the sample exposure. The baseline data for a functionalized sensor can be collected using the apparatus for analyte detection described above. In some embodiments, the bassline data can be collected prior to exposure of the sensor to the sample. In some embodiment, the baseline data is provided for a sensor functionalized with a specific biological probe. A shift in the spectral peaks after the sample exposure indicates the binding of the target analyte with the biological probe, therefore indicating the presence the target analyte in the sample. In some embodiments, the amount of the spectral peak shift may further be interpreted to provide a quantitative or semi-quantitative measurement of the concentration of a target analyte in the sample.

In some embodiments where the sensors in the array are functionalized with different biological probes, exposure of the array to the sample can result in binding of various target analytes to the corresponding sensors. Illuminating the array of sensors with a light at a series of wavelengths would allow the collection of optical spectra of each sensor be collected and compared with the baseline data. One exposure of the sensing device chip could allow detection and identification of different target analytes.

In some embodiments, the plasmon-resonance sensing device enables point-of-care (POC) detection of target analytes and POC diagnosis of disease(s)/condition(s). In some embodiments, rapid results (about 15 min or less) may be provided.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms “comprising,” “including,” “containing,” and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

The term “nanostructure,” as used herein, has its standard scientific meaning and thus refers to any structure that is between about molecular size, to about microscopic size. Nanostructures comprise nanomaterials, which can be any material in which a single unit is sized at about 1 nm to about 200 nm. Nanostructures include nanoparticles, nanorods, nanosquares, nanocubes, gradient multilayer nanofilm (GML nanofilm), icosahedral twins, nanocages, magnetic nanochains, nanocomposite, nanofabrics, nanofiber, nanoflower, nanofoam, nanohole, nanomesh, nanopillar, nanopin film, nanoplatelet, nanoribbon, nanoring, nanobipyramids, irregular nanoparticles, nanosheet, nanoshell, nanotip, nanowire, and nanostructured film. It will be understood that a nanostructure can have various geometric shapes and properties based on the components of that nanostructure.

The term “analyte” refers to a substance or chemical constituent that is of interest. For examples, analyte may include biological or chemical substance that may be detected by a sensing device and may be of interest for diagnosing a disease or a condition.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### Examples

The technology described herein is further illustrated by the following examples which in no way should be construed as being further limiting.

### Example 1: Electromagnetic Simulation

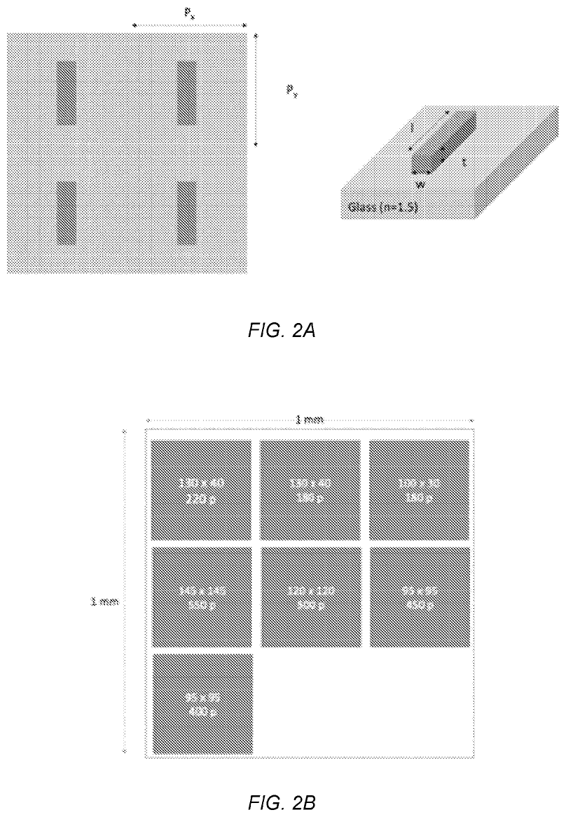

Several geometries for simulation and testing included some nanorods and some coupled nanoarrays. The nanorods is designed to reflect randomly oriented colloidal nanorods dispersed onto a glass slide. The coupled nanoarrays are designed to create surface lattice resonances. The seven geometries for a fabrication dose test are as shown in Table 1 and FIGS. 2A and 2B. FIG. 2A shows a grid with labeled dimensions for length (l), width (w), thickness (t), and spacing/periodicity (p) of the nanorods. FIG. 2B is a map of arrangement of the nanorod array within a sensor unit. As show in Table 1, the test geometries T1-T3 are nanorods and the test geometries T4-T10 are coupled nanoarrays.

TABLE 1
Test Geometries. Length, width, periodicity, and thickness of
the dose matrix test. All dimensions are listed in nanometers.

| | Length | Width | Thickness | Spacing |
|---|---|---|---|---|
| Sample | (l), nm | (w), nm | (t), nm | (p), nm |
| T1 | 130 | 40 | 40 | 220 |
| T2 | 130 | 40 | 40 | 180 |
| T3 | 100 | 30 | 40 | 180 |
| T4 | 145 | 145 | 40 | 550 |
| T5 | 120 | 120 | 40 | 500 |
| T6 | 95 | 95 | 40 | 450 |
| T7 | 95 | 95 | 40 | 400 |
| T8 | 95 | 95 | 50 | 450 |
| T9 | 120 | 120 | 40 | 500 |
| T10 | 145 | 145 | 40 | 550 |

Full-wave electromagnetic simulations were conducted using Lumerical photonic simulation software. Periodic boundary conditions were applied in the x- and y-dimensions for each of the geometries T1-T7 as shown in FIGS. 2A-2B. For bulk sensing experiments, the refractive index of the surrounding media was changed. For PNA-DNA binding experiments, conformal shell layers of defined refractive index were modeled atop the nanostructures. Extinction and transmittance curves were returned for the wavelength range of 400-1200 nm.

### Example 2: Simulation Setup and Defining Figure-of-Merit

To study the plasmonic resonance shape as well as sensitivity to changes in refractive index, initial simulations included a bulk refractive index sensitivity analysis. In an initial iteration, gold nanorods with a wide spacing designed to represent the ordered nanoarrays was tested.

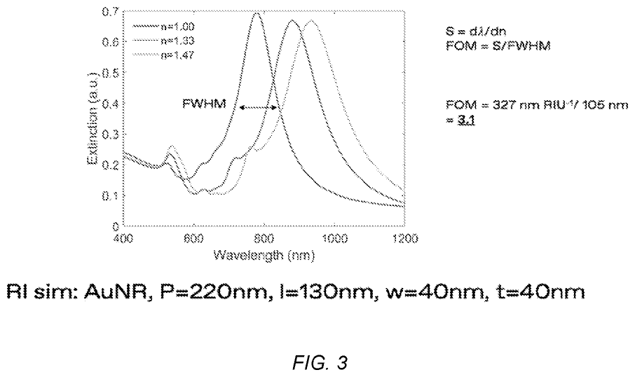

The resonances were modeled in air, water, and glycerol (increasing refractive index) and the peak locations were calculated for each of the extinction curves. This allowed for development of a sensor figure-of-merit (FOM) that considers the peak shift(s) and the narrowness of the resonances (full width at half maximum-FWHM) as shown in FIG. 3. The figure of merit was defined as the shift over full width at half maximum, allowing for a direct comparison between various geometries. A larger figure of merit represents better sensing performance due to (1) larger peak shifts for the same refractive index change, and (2) easier discrimination of peak shifts due to a narrow resonance curve. This analysis was repeated for all geometries considered.

### Example 3: PNA-DNA Binding Simulation

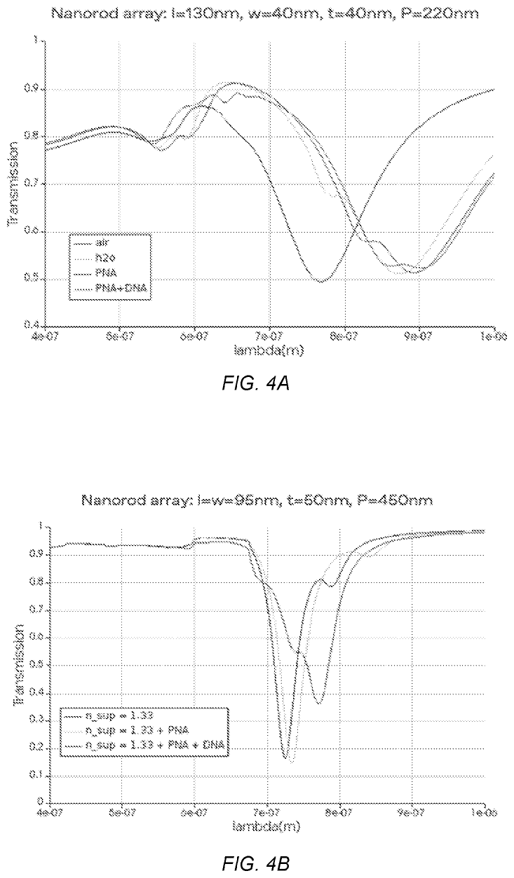

Another method of simulating these nanostructures involved simulating conformal layers with the same refractive indices expected of peptide nucleic acid (PNA) probes and PNA probes bound to DNA. We observed that the shift upon PNA+DNA binding for the surface lattice geometry (as shown in FIG. 4B) is much more apparent than the shift for the disperse nanorod geometry (as shown in FIG. 4A). These simulations point to expected shifts associated with DNA biosensing for each geometry.

### Example 4: Nanosensor Fabrication

Electron-beam lithography is a common method for patterning precise nanoscale features onto a substrate. Typically, such patterns are processed onto silicon wafers, which are optically opaque and highly conductive. For the transmittance-mode operation of the sensor, the nanostructures were configured to sit atop a transparent quartz wafer. A protocol for nanoscale patterning onto a transparent, non-conductive surface was developed.

First a thin layer of a conductive photoresist was spin-coated on the transparent quartz wafer before exposure to the pattern with an electron beam (JEOL E-beam microscope). After this, a thin (~5 nm) chromium adhesion layer was thermally evaporated onto the patterned substrate, followed by a thicker (about 40-50 nm) pure gold layer. Chemical liftoff was conducted to form the nanostructures array before dicing the substrate for testing. The first sample produced with this pattern was a dose matrix test to evaluate the power of the electron beam. After this parameter was identified, all future processes were conducted under the same conditions.

### Example 5: Simulation of Selected Geometries

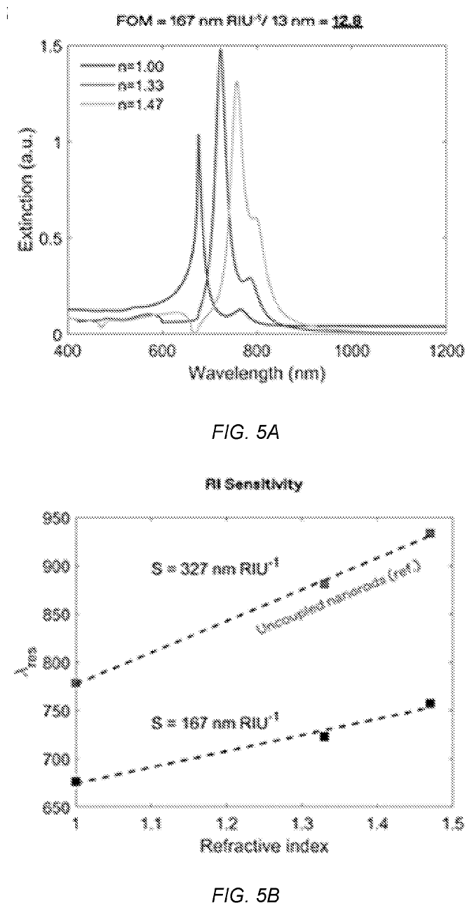

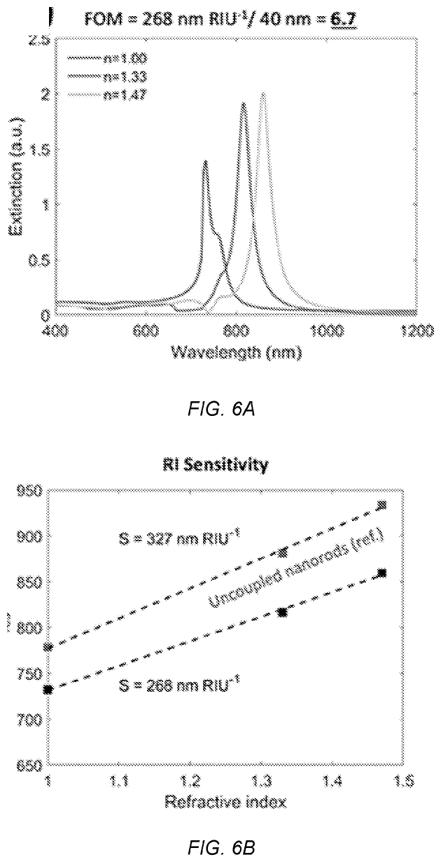

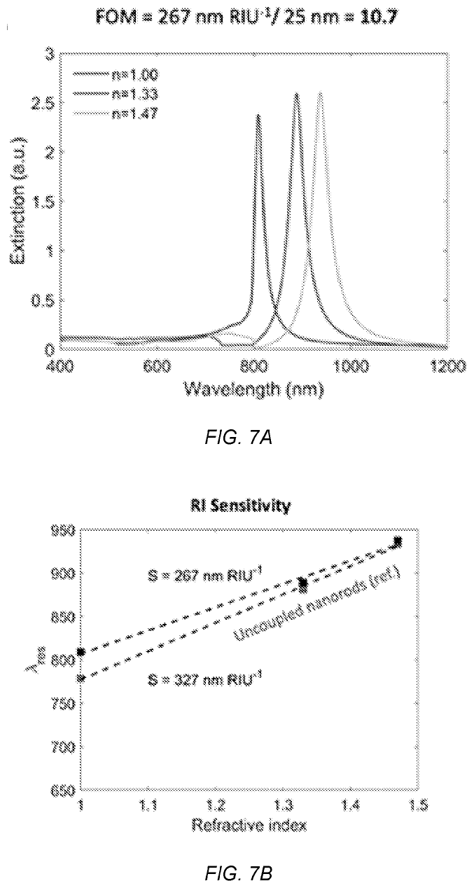

Bulk refractive simulations were conducted on sample geometries T8-T10 described in Table 1. Transmittance through the samples was measured using an optical readout instrumentation. Wavelength bounds were set from 450 nm to 950 nm. For seamless integration with the readout instrumentation, the individual sensor of the sensing device was fabricated to have a 1 mm² area of nanostructures array to fully align with the light source spot size and minimizing signal loss. The results for the three surface lattice resonance geometries (T8-T10) are as shown in FIGS. 5A/B, 6A/B, and 7A/B, respectively. Both the shape of the peak and the refractive index peak shifts are shown. The calculated figure-of-merits for T8-T10 were 12.8, 6.7, and 10.7, respectively. Further, the refractive index sensitivities of each of these geometries are shown in FIGS. 5B, 6B, and 7B. All sensitivities are compared to the 140 nm×40 nm 220p sample labeled “uncoupled nanorods”. A higher slope indicates better sensing performance. Sample geometry T10 is the highest performance due to its high figure of merit (10.7) and its relatively high refractive index sensitivity (267 nm/RIU).

### Example 6: Comparison of Simulation and Experiment

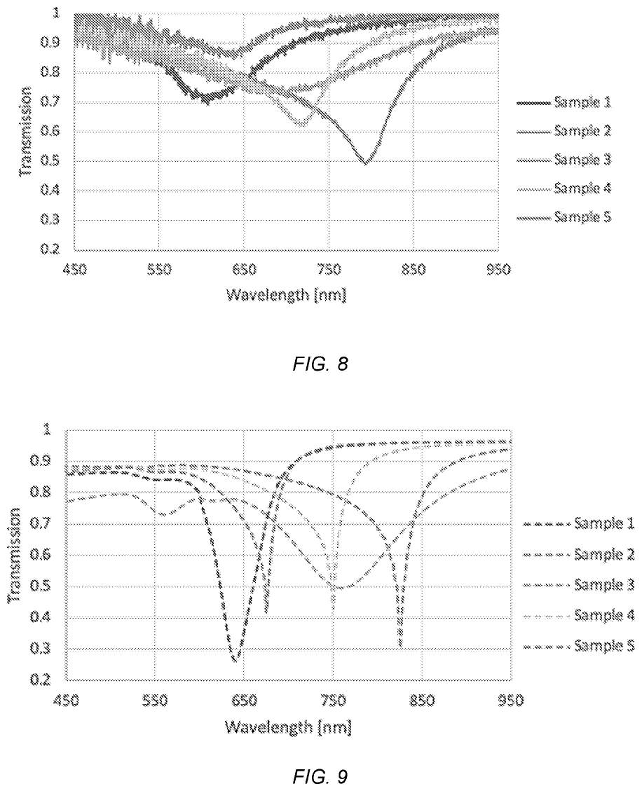

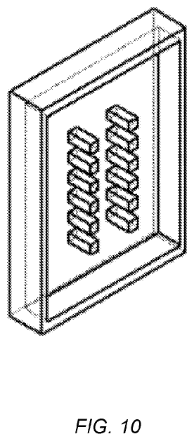

Nanostructure array samples 1-5 were fabricated with the nanostructure dimensions shown in Table 2. The transmittance of each sample was experimentally measured (shown in FIG. 8) and compared to the peak shape from the simulations (shown in FIG. 9). There was found to be exceptional agreement between the experimental and simulation data, including the peak shape and resonance location.

TABLE 3
Dimensions of nanostructure arrays.

| | Length | Width | Thickness | Spacing |
|---|---|---|---|---|
| Sample | (l), nm | (w), nm | (t), nm | (p), nm |
| 1 | 96 | 96 | 50 | 400 |
| 2 | 145 | 145 | 50 | 550 |
| 3 | 130 | 130 | 50 | 220 |
| 4 | 120 | 120 | 50 | 500 |
| 5 | 95 | 95 | 50 | 450 |

The present disclosure also puts forth a methodology for rational design of regularly spaced nanoparticle arrays for plasmonic sensing. The Applicants tested 5-7 geometries through both simulation and experimental analysis, and finally selected the 145 nm×145 nm Through both simulation and experimental analysis, a nanoarray geometry that shows high-amplitude resonance and refractive index sensitivity may be selected for the production of the plasmonic-resonance sensing device.

### Example 7: Functionalization of Nanostructures

A 2×6 array of 1 mm² sensors (12 sensors total) was functionalized with peptide-nucleic acid (PNA) probes. Each of the sensors contains an array of 145 nm×145 nm gold nanostructures with regular spacing. In order to individually functionalize the sensor arrays to be target-specific, a polydimethylsiloxane (PDMS) polymer micro-well array was fabricated. This micro-well array was aligned with the substrate such that each sensor could be accessed through a single micro-well. This approach created repeatable, programmable coordinates for the automatic pipetting system (e.g., Integra ASSIST PLUS pipetting robot).

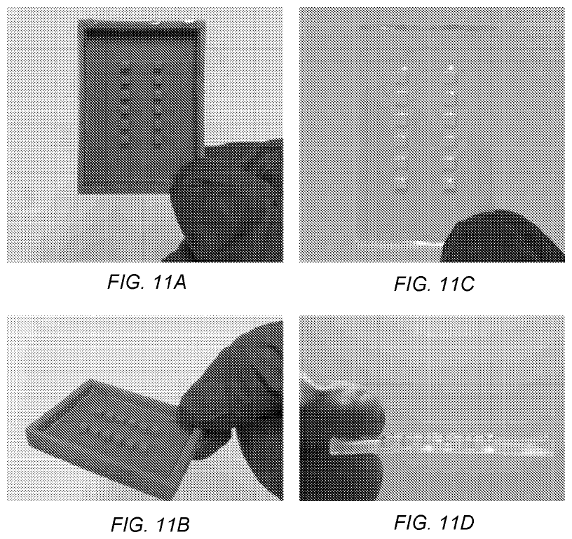

The micro-well structure atop the sensing array allowed for individual fluid delivery to each sensing spot, enabling multiplexing of up to 12 targets on a single sensing chip. To this end, a mold was designed using Solidwaorks CAD to allow for fabrication of a polymer micro-well array that align with the coordinates of the sensors (FIG. 10). The mold for casting the PDMS micro-wells was designed in Solidworks consisting of twelve 2 mm×2 mm×5 mm (20 mm³) pillars. The pillars were positioned to match the coordinates of sensor array on the glass substrate. Master molds, as shown in FIGS. 11A and 11B, were then made using SLA 3D printing.

Micro-well array devices were fabricated in the molds using PDMS soft lithography. Sylgard 184 silicone elastomer, base and curing agent (Dow Corning, Midland, MI) were mixed in a ratio of 10:1, by weight. Next, the PDMS prepolymer was cast on the master mold and cured at 80° C. in a convection oven for approximately 1.5 h. The cured PDMS micro-well array, as shown in FIGS. 11C and 11D, was removed from the master mold. The polymer micro-well array was affixed atop the sensor array using washable glue, enabling removable bonding for sensor reuse. This entire system was attached to a standard 75×25 mm microfluidic chip and was then ready for molecular detection.

### Example 8: Automated Robotic Functionalization of Sensors

The prepared plasmonic sensing chip was integrated with the automatic pipetting system (e.g., Integra ASSIST Plus) for surface functionalization. To covalently functionalize the sensor with selected biological probes, such as PNA probes, the gold nanostructures on a glass substrate were first incubated with 1 mg/mL dithiobis succinimidyl propionate (DSP) dissolved in dimethyl sulfoxide (DMSO) for 20 min. This crosslinking molecule activated the gold surface to enable coupling of free amines on the PNA. Next, the sensor arrays were put in contact with 1 mg/mL PNA probe dispersed in Tris-EDTA buffer (pH 7.0) for 30-45 min. Transmission spectra were collected before and after conjugation to characterize successful PNA conjugation.

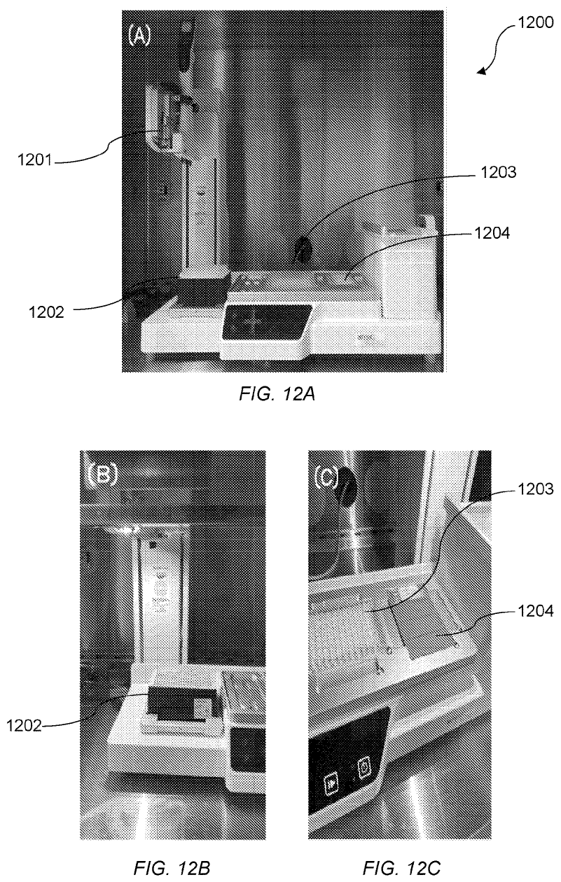

The sensor functionalization process described above was automated using an Integra ASSIST PLUS pipetting robot. In order to effectively position the device onto the deck of the Integra ASSIST PLUS pipetting robot, we designed and fabricated (3D printed) a custom 4-slot microscope slide holder/adaptor the size of a standard 96-well plate. This adapter could readily be integrated with the liquid handler's robotic deck. A 96-well plate was pre-loaded with functionalization reagents and placed in the robot's aspiration deck. To start up the machine, a Voyager electronic 125 μL, 8-channel pipette was loaded onto the robot. A suite of six programs were developed to aspirate, dispense, and clear tips in an automated fashion. These custom programs allow for multiplexed functionalization of twelve PNAs upon the sensor arrays. Table 4 shows 6 programs for automated functionalization of the sensors using the pipetting robot. The programs indicate pipette tip location, 96-well plate location, aspiration volume, and dispense volume for each stage. FIG. 12A is a photo of the Integra ASSIST PLUS pipetting robot 1200, with pipette holder 1201 on the left, tip box 1202, 96 -well plate holder 1203, and custom chip adapter 1204. FIG. 12B depicts the tip box 1202 aligned under pipette holder 1201. FIG. 12C depicts the 96 well plate 1203 and adapter 1204 during functionalization.

TABLE 4
Integra ASSIST custom programs.

| | | | | | Dispense |
|---|---|---|---|---|---|
| | | | 96 Well Palate | Application | Volume (μL)/ |
| # | Program Name | Pipette Tip Location | Location | Volume (μL) | Sensor Well |
| 1 | Dispense TE | A1, B1 | A1, B1 | 80 | 12 |
| 2 | Aspirate TE | I1, J1 | X | 12 | X |
| 3 | Dispense DSP | A2, B2 | A2, B2 | 80 | 12 |
| 4 | Aspirate DSP | I2, J2 | X | 12 | X |
| 5 | Dispense PNA | A, B(3-5); I, J(3-5) | A(3-8), B(3-8) | 15 | 12 |
| 6 | Aspirate PNA | A, B(6-8); I, J(6-8) | X | 12 | X |

First, the Tris-EDTA (TE) buffer is dispensed and removed from the chip surface to clean the surface and to ensure a tight seal of the micro-well array onto the sensing substrate. Then DSP, a bivalent cross-linking molecule, is introduced to the chip surface and readily adsorbs to the gold surface within 15-20 minutes. The presence of active NHS groups enables cross-linking to proteins (i.e., PNAs). Examples of linkers for attaching a capturing ligand/biological probe (such as PNA) are presented in Table 5. Finally, the DSP is aspirated and the PNA probes are directly dispensed atop the sensing surface and couple to the free amines on the nanostructures. After the excess PNA solution is aspirated, the chip is covalently functionalized with PNAs and ready to use for sample testing.

TABLE 5
Attachment of Ligand to Gold.

| | |
|---|---|
| Active Group | Cross-linking molecules for conjugation to gold |
| on Ligand | |
| Amine | DSP (dithiobis(succinimidyl propionate)) |
| Cysteine | Direct attachment of thiol group |
| Thiol | Direct attachment of thiol group |
| Amine | EDC (1-ethyl-3-(3-dimethylaminopropyl)carbodiimide |
| | hydrochloride) and NHS (N-hydroxysulfosuccinimide) |
| Amine | p-mercaptobenzoic acid (p-MBA) self-assembled |
| | monolayer followed by EDC and NHS |

While pipetting robot systems have been widely utilized for fluid loading applications, to the best of our knowledge this is the first time such a system has been employed for covalently attaching a molecular capture probe to a solid-state sensor. We accomplish this through successive dispense and aspiration steps atop the sensors.

The scope of the present disclosure is not intended to be limited by the specific disclosures of examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A plasmon-resonance sensing device comprising:
an array of sensors, each sensor comprises an array of nanomaterials that are regularly spaced apart with a spacing of from about 100 nm and about 2000 nm between the nanomaterials;
wherein each of the nanomaterials has a square shape with a side dimension of from about 50 nm to about 400 nm; and
the nanomaterials are conjugated with a biological probe configured to bind to an analyte.

2. The plasmon-resonance sensing device of claim 1, wherein the nanomaterials have a thickness of from about 20 nm to about 75 nm.

3. The plasmon-resonance sensing device of claim 1, wherein the nanomaterials comprise gold.

4. The plasmon-resonance sensing device of claim 1, wherein the biological probe is selected from the group consisting of a peptide-nucleic acid, an aptamer, an antibody, an antibody fragment, a complimentary DNA, and an enzyme.

5. The plasmon-resonance sensing device of claim 1, wherein at least a first sensor in the array of sensors comprises nanomaterials conjugated with a first biological probe and at least a second sensor in the array of sensors comprises nanomaterials conjugated with a second biological probe.

6. A method for detecting an analyte in a sample comprising:
exposing at least one sensor in the plasmon-resonance sensing device of claim 1 to the sample, wherein the analyte in the sample binds to the at least one sensor;
heating the at least one sensor after exposing the at least one sensor to the sample;
illuminating a light at a series of wavelengths onto the at least one sensor; and
collecting absorbance, transmittance, or extinction data of the sensor.

7. The method of claim 6, further comprising flowing a plurality of functionalized particles over the at least one sensor after exposing the at least one sensor to the sample, wherein the plurality of functionalized particles is configured to bind to the analyte that is bound to the at least one sensor.

8. The method of claim 6, further comprising comparing the collected absorbance, transmittance, or extinction data with a baseline data of the sensor prior to the sample exposure.

9. The method of claim 6, wherein an array of sensors in the plasmon-resonance sensing device of claim 1 is exposed to the sample.

10. The method of claim 9, wherein at least a first sensor in the array of sensors comprises nanomaterials conjugated with a first biological probe and at least a second sensor in the array of sensors comprises nanomaterials conjugated with a second biological probe.

11. The method of claim 10, wherein the first biological probe and the second biological probe are independently selected from the group consisting of a protein-nucleic acid, an aptamer, an antibody, an antibody fragment, a complimentary DNA, and an enzyme.

12. The method of claim 11, wherein the first biological probe and the second biological probe are different.

13. A method of making an array of nanomaterials comprising:
coating a conductive photoresist layer onto a non-conductive substrate;
patterning the conductive photoresist layer via lithography, thereby forming a patterned substrate;
depositing an adhesion layer onto the patterned substrate; and
depositing a metallic layer onto the adhesion layer.

14. The method of claim 13, wherein the metallic layer comprises gold.

15. The method of claim 13, wherein the metallic layer has a thickness of about 20 nm to about 75 nm.

16. The method of claim 13, wherein the adhesion layer comprises chromium.

17. The method of claim 13, wherein the adhesion layer has a thickness of about 5 nm.

18. The method of claim 13, wherein the nanomaterials are regularly spaced apart with a spacing of from about 100 nm and about 2000 nm in between, and each of the nanomaterials has a square shape with a side dimension of from about 50 nm to about 400 nm.

19. A method of making a functionalized nanoplasmonic sensing chip comprising:
providing a substrate comprising an array of sensors;
affixing a micro-well adaptor on top of the substrate, thereby providing an array of micro-wells over and aligned with the array of sensors;
forming one or more functionalized sensors in the array of sensors comprising:
delivering a first batch of reaction solutions into one or more micro-wells atop one or more sensors;
subsequently removing the first batch of reaction solutions from the one or more micro-wells;
wherein the delivering and removing of the first batch of reaction solutions are performed by an automatic pipetting system; and
removing the micro-well adaptor from the substrate.

20. The method of claim 19, wherein the first batch of reaction solutions comprises two or more different reaction solutions.

21. The method of claim 19, wherein the forming one or more functionalized sensors further comprises delivering a second batch of reaction solutions into the one or more micro-wells, and subsequently removing the second batch of reaction solutions from the one or more micro-wells, wherein the delivering and removing of the second batch of reaction solutions are performed by the automatic pipetting system.

22. The method of claim 21, wherein the second batch of reaction solutions comprises two or more different reaction solutions.

23. The method of claim 19, wherein the one or more functionalized sensors comprises one or more biological probes.

24. The method of claim 23, wherein each of the one or more biological probe are independently selected from the group consisting of a peptide-nucleic acid, an oligonucleotide, an aptamer, an antibody, an antibody fragment, a complimentary DNA, and an enzyme.

25. The method of claim 23, wherein the one or more biological probes are the same.

26. The method of claim 23, wherein all of the one or more biological probes are different.

27. The method of claim 19, wherein the first batch of reaction solutions are delivered to the one or more micro-wells simultaneously.

28. A functionalized nanoplasmonic sensing chip comprising an array of functionalized sensors, wherein each of the functionalized sensors in the array comprises an array of nanomaterials conjugated to at least one biological probe configured to bind to at least one analyte.

29. The functionalized nanoplasmonic sensing chip of claim 28, wherein the array of nanomaterials is conjugated to two or more biological probes configured to bind to two or more analytes.

30. The functionalized nanoplasmonic sensing chip of claim 28, wherein the at least one biological probe is independently selected from the group consisting of a peptide-nucleic acid, an oligonucleotide, an aptamer, an antibody, an antibody fragment, a complimentary DNA, and an enzyme.

31. The functionalized nanoplasmonic sensing chip of claim 28, wherein at least one of the functionalized sensors in the array comprises at least one different biological probe from the others.

32. The functionalized nanoplasmonic sensing chip of claim 31, wherein each of the functionalized sensors in the array comprises at least one different biological probe.

33. The functionalized nanoplasmonic sensing chip of claim 28, wherein the array or nanomaterials comprises gold.

34. The functionalized nanoplasmonic sensing chip of claim 28, wherein the nanomaterials in the array are regularly-spaced apart with a spacing of from about 100 nm and about 2000 nm, and each nanomaterials has a square shape with a side dimension of from about 50 nm to about 400 nm.

35. The functionalized nanoplasmonic sensing chip of claim 34, wherein the nanomaterials have a thickness of from about 20 nm to about 75 nm.

36. A method for detecting two or more analytes in a sample simultaneously comprising:
exposing the array of functionalized sensors on the functionalized nanoplasmonic sensing chip of claim 32 to the sample;
heating the array of functionalized sensors;
illuminating a light at a series of wavelengths onto each of the functionalized sensors; and
collecting absorbance, transmittance, and/or extinction data of each functionalized sensor.

37. The method of claim 36, further comprises comparing the collected absorbance, transmittance, and/or extinction data of each functionalized sensor with a baseline data of each of the functionalized sensors prior to the sample exposure.

38. The method of claim 36, wherein up to 50 analytes in the sample are detected.
